(19) United States
(12) Patent Application Publication
(10) Pub. No.: US 2012/0127433 A1
(43) Pub. Date: May 24, 2012

(54) SELF-SCREENING WELLNESS ASSESSMENT AND RECOMMENDATION SYSTEM

(75) Inventors: Jorlin E. Moon, Los Altos, CA (US); David L. Carlberg, Sonora, CA (US); Alec W. Kercsó, Atherton, CA (US)

(73) Assignee: FirstPoint Health, Inc., Menlo Park, CA (US)

(21) Appl. No.: 13/304,253

(22) Filed: Nov. 23, 2011

Related U.S. Application Data
(60) Provisional application No. 61/417,190, filed on Nov. 24, 2010.

(51) Int. Cl. A61B 3/032; A61B 5/00

(52) U.S. Cl. 351/223; 600/300

(57) ABSTRACT

A self-screening wellness assessment and recommendation system is described, including a device having monitors configured to display graphics associated with vision screening, mirrors configured to reflect graphics along an optical path, the mirrors enclosed in a moveable chamber, a view portal opening through which graphics are visible, a display configured to provide a user interface associated with a wellness assessment, a touchscreen configured to transmit a user selection to a computer, and a housing. In some examples, a method using a self-screening wellness assessment and recommendation system may include positioning a user interface at an eye level, the user interface being displayed on a monitor coupled to a lift, requesting input associated with a wellness assessment using the user interface, performing the wellness assessment, providing a result of the wellness assessment using a display, and providing a recommendation associated with the result on the display.

### CROSS REFERENCE TO RELATED APPLICATIONS

This application is a U.S. non-provisional patent application that claims the benefit of U.S. Provisional Patent Application No. 61/417,190 (Attorney Docket No. MOO-001P), filed Nov. 24, 2010, and entitled “Self-Screening Health Assessment,” which is herein incorporated by reference for all purposes.

### FIELD

The present invention relates generally to health care. More specifically, techniques described relate to a self-screening wellness assessment and recommendation system.

### BACKGROUND

Many people, in both developing and developed countries, do not have reliable and ready access to complete health and vision care. Even for people that do have access to medical and vision care, many of these people do not know, and do not have the tools to determine, when and if to seek the help of a medical or eye care professional. This is a widespread problem given the numerous trends, including technological, environmental, and sociological, that are threatening the health and wellness of adults and children in both the developed and developing countries. Vision and eye health are often neglected, which is problematic as the stress load on eyes has never been greater with the proliferation of computers, cell phones, the internet, iPods, iPads, portable DVD players, and 3-D movies. It is known that vision and eye health change with age. However, the impact is yet to be measured of both aging and a life of increasing demands on people's eyes caused by the technology we use every day. The lack of access to proper medical care, increased exposure to ultraviolet light in the environment due to a thinning ozone layer, environmental pollutants, smoking, poor diet, obesity, and lack of exercise, among other factors, all contribute to the problem of health and vision degradation, and eventually to the possibility of loss of sight and onset of serious diseases.

Important factors that also contribute to ocular diseases and sight loss include the global lack of knowledge concerning the symptoms of common vision and eye conditions, and infrequent or absence of a thorough eye exam by an eye care professional. The problem is further exacerbated by the often unperceivable slow changes to vision acuity and the common belief that one's vision is fine when actually it has deteriorated. Many serious eye conditions do not exhibit symptoms until they are in advance stages of the disease. Early detection and treatment of vision health issues by a trained eye care professional lead to better outcomes and lower health care costs.

Diabetes is a group of diseases where patients exhibit a high level of blood glucose resulting from a faulty insulin production system. Diabetes is a leading cause of new cases of blindness, and is ranked the seventh leading cause of death in the United States. Diabetic related blindness is increasing due to the lack of information among diabetics about the seriousness of the disease, the continual need to monitor and control their insulin levels, and the necessity of seeing an eye care professional on a frequent basis. Tens of millions of Americans have diabetes, both diagnosed and undiagnosed. The annual direct medical costs association with diabetes is estimated to be over $100 Billion, and the indirect costs that include work loss, disability, and premature mortality, and blindness are estimated at an additional $50+ Billion.

Heart conditions also have been linked to vision and eye health. Medical science has recognized that what is good for the heart also is good for one's vision and eye health. A particular condition known as hypertension (i.e., high blood pressure) can lead to heart and kidney disease, stroke, and blindness. It is estimated that about two-thirds of people over the age of 65 have high blood pressure. Those people who do not have high blood pressure by the age of 55 have a nearly 90 percent chance of developing it during their lifetimes. High blood pressure most often can be controlled through diet, exercise, and medication. However, there are many persons with high blood pressure that are unaware of it, and do not know the seriousness of the effects of the disease, particularly its impact on vision.

Conventional self-screening health assessment devices exist, but are often relegated to assessing one aspect of a user's health. Some conventional devices are able to assess a person's blood pressure or vision, but not both. Conventional devices also are unable to provide wellness recommendations based upon multiple aspects of a person's overall health. Given the serious health, vision and eye health issues that do not present easily detected symptoms, it is important for people to have access to more comprehensive self-screening wellness assessments to determine when and if to seek the help of a medical or eye care professional.

Thus, what is needed is a solution for a self-screening wellness assessment and recommendation system without the limitations of conventional techniques.

### BRIEF DESCRIPTION OF THE DRAWINGS

Various embodiments of the invention are disclosed in the following detailed description and the accompanying drawings:

FIGS. 1A-1B illustrate front and side views, respectively, of an exemplary device for implementing a self-screening wellness assessment and recommendation system;

FIGS. 2A-2B illustrate front and side views, respectively, of an alternative exemplary device for implementing a self-screening wellness assessment and recommendation system;

FIGS. 3A-3C illustrate front, top and bottom views, respectively, of an exemplary optical chamber in an exemplary device for implementing a self-screening wellness assessment and recommendation system;

FIGS. 4A-4C illustrate front, right side and left side views, respectively, of an alternative exemplary optical chamber in an exemplary device for implementing a self-screening wellness assessment and recommendation system;

FIGS. 5A-5B illustrate exemplary processes for providing a self-screening wellness assessment and recommendation;

FIGS. 6A-6H illustrate wireframes of exemplary interfaces associated with a self-screening wellness assessment and recommendation system;

FIG. 7A illustrates an exemplary computer system suitable for implementation of a self-screening wellness assessment and recommendation system; and

FIG. 7B illustrates an alternative exemplary computer system suitable for implementation of a self-screening wellness assessment and recommendation system.

### DETAILED DESCRIPTION

Various embodiments or examples (hereafter “examples”) may be implemented in numerous ways, including as a system, a process, an apparatus, a user interface, or a series of program instructions on a computer readable medium such as a computer readable storage medium or a computer network where the program instructions are sent over optical, electronic, or wireless communication links. In general, operations of disclosed processes may be performed in an arbitrary order, unless otherwise provided in the claims.

A detailed description of one or more examples is provided below along with accompanying figures. The detailed description is provided in connection with such examples, but is not limited to any particular example. The scope is limited only by the claims and numerous alternatives, modifications, and equivalents are encompassed. Numerous specific details are set forth in the following description in order to provide a thorough understanding. These details are provided for the purpose of example and the described techniques may be practiced according to the claims without some or all of these specific details. For clarity, technical material that is known in the technical fields related to the examples has not been described in detail to avoid unnecessarily obscuring the description.

In some examples, the described techniques may be implemented as a computer program or application (“application”) or as a plug-in, module, or sub-component of another application. The described techniques may be implemented as software, hardware, firmware, circuitry, or a combination thereof for purposes of providing computational and processing capabilities. If implemented as software, the described techniques may be implemented using various types of programming, development, scripting, or formatting languages, frameworks, syntax, applications, protocols, objects, or techniques, including C, Objective C, C++, C#, Adobe® Integrated Runtime™ (Adobe® AIR™), ActionSript™, Flex™, Lingo™, Java™, Javascript™, JSON, Ruby, Rails, Ajax, Perl, COBOL, Fortran, ADA, XML, MXML, HTML, DHTML, XHTML, HTTP, XMPP and others. Also, if implemented as software, the described techniques may be implemented using a software development kit (SDK) or application programming interface (API). Design, publishing, and other types of applications, such as Dreamweaver®, Shockwave®, Flash®, and Fireworks®, may also be used to implement the described techniques. The described techniques may be varied and are not limited to the examples or descriptions provided.

As described herein, techniques associated with a self-screening wellness assessment and recommendation system are described, including providing recommendations, advertisements or incentives (e.g. discounts on products) associated with a user's wellness assessment. Health and wellness (hereinafter collectively referred to as “wellness”) assessments may be performed, and the results presented, on devices as described herein. As used herein, the terms “assessment” and “screening” are used interchangeably to refer to an evaluation, and a wellness assessment comprises an evaluation of various aspects of a user's wellness, including a user's vision acuity (e.g., distance, near and central vision acuity), eye health (e.g., eye dryness, ocular allergies, cataracts, diabetic retinopathy, glaucoma, macular degeneration, ocular sun damage, etc.), flu risk, diabetes risk, body mass index (BMI), risk of cardiovascular disease, and other health risks. In some examples, the evaluation may be performed by eliciting information (e.g., by asking questions) about a user that may be useful to evaluating such health risks (e.g., age, sex, race or ethnicity, height, weight, waist size, smoking and drinking habits, medical history, vision acuity assessment, etc.). Results and recommendations associated with the wellness assessments also may be communicated to a user or to a remote location using the internet, mobile phone, or other methods of data communications. Further screening, analysis, and progress may also be performed and tracked using a website, web application, downloadable and/or mobile application, associated with the self-screening wellness assessment and recommendation system. The system may be configured to provide information on health conditions and diseases that are age, gender, ethnic, and lifestyle related, and products and services that assist users with improving and preserving their ocular, heart, and overall health. The system also may be configured to correlate various wellness assessments described herein, and provide information regarding such correlations between various health conditions, the impact they may have upon each other, as well as how they may impact the health of the user now and as the user ages. Optional rewards and incentives may be implemented as part of the system to motivate the user to achieve user-set health goals.

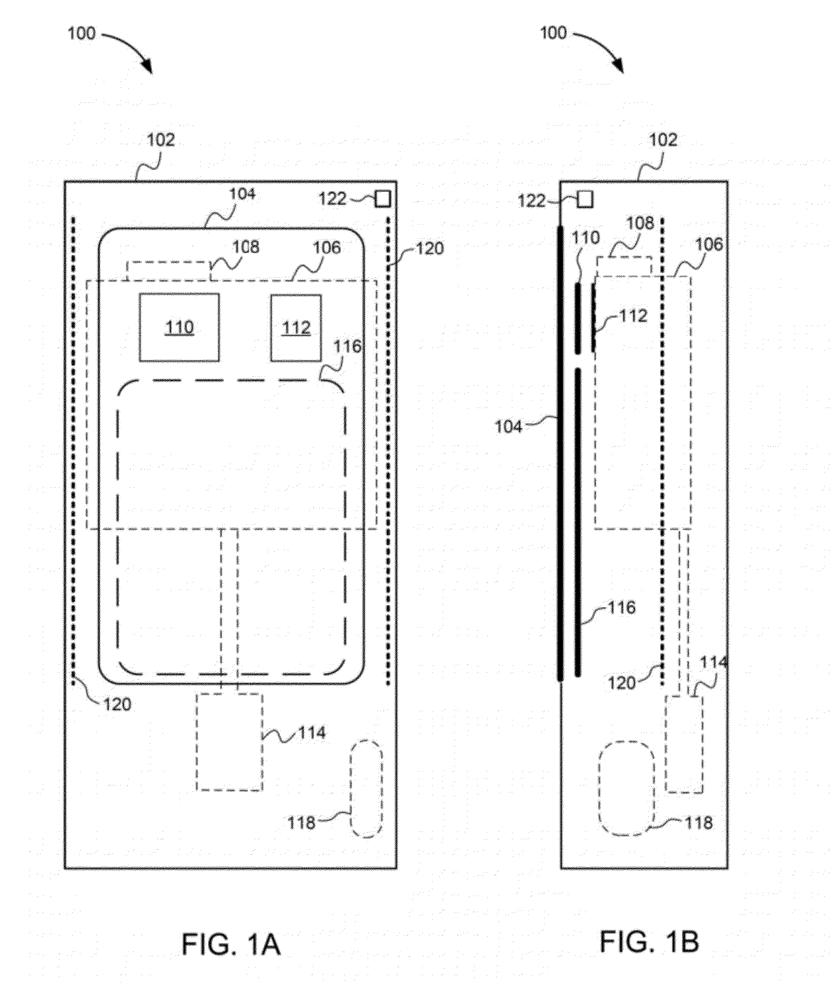

FIGS. 1A-1B illustrate front and side views, respectively, of an exemplary device for implementing a self-screening wellness assessment and recommendation system. FIG. 1A shows a front view, and FIG. 1B shows a side view, of device 100. Like-numbered and named elements in these views may describe the same or substantially similar elements. As shown, device 100 includes housing 102, touchscreen 104, chamber 106, monitors 108 and 110, view portal 112, lift 114, display 116, computer 118, rails 120, and sensor 122. In some examples, housing 102 may be configured to cover and support the internal components of device 100. In some examples, housing 102, or parts of housing 102, may be formed from various types of materials, including different kinds of metal, metal alloy, plastic, rubber, and other materials. For example, housing 102 may be composed of steel, alloy steel, aluminum or aluminum alloy. In some examples, housing 102 may include a door (not shown) or other means of opening device 100 for ease of access to the parts of device 100 housed within housing 102, for example, for repair or maintenance. For example, the door may comprise a front half of the housing, or part of the front half of the housing, with a latch on one side and a hinge on the other side. In an example, the latch may be secured by a locking mechanism. In another example, touchscreen 104 may be coupled to or mounted on the front of housing 102.

In some examples, touchscreen 104 may be configured to enable a user to interact with a displayed interface (e.g., provided using display 116) without the use of an intermediary input device or cursor controller (e.g., separate keyboard, mouse, trackball, etc.). In some examples, a user may view graphics or images (e.g., a user interface, advertising, information, coupons, etc.) shown by display 116 through touchscreen 104, and touchscreen 104 may detect or sense the presence and location of a touch within a display area associated with the user interface or other graphic shown on display 116. In some examples, display 116 may be implemented as a monitor, screen, or other device operable to show graphics and images (e.g., CRT, LED, LCD, plasma, OLED, etc.). In some examples, touchscreen 104 may use infrared (“IR”), resistive, surface acoustic wave, surface capacitance, projected capacitance, mutual capacitance, optical imaging, dispersive signal, acoustic pulse recognition, or other technologies. For example, touchscreen 104 may be an IR touchscreen that uses IR light-emitting diodes (LEDs) and sensors (e.g., photodetector, photo transistor, etc.) to detect a disruption in a pattern of LED beams (e.g., LED beams that cross each other in vertical and horizontal patterns), which enables the sensors to pinpoint the exact location of a touch. In some examples, touchscreen 104 may be configured to detect any type of touch (e.g., by a finger, gloved finger, stylus, pen, etc.).

In some examples, touchscreen 104, display 116 and computer 118 may be configured to communicate with each other. In other examples, computer 118 also may communicate with lift 114, monitors 108 and 110, sensor 122 and/or other parts of device 100. In some examples, connectivity may be implemented using various data communication techniques (e.g., using a network interface card (NIC) to transmit and receive data packets over a public or private data network, (e.g., the Internet, a private local area or wide area network (i.e., LAN or WAN), etc.). Each of computer 118, display 116, monitors 108 and 110, sensor 122, and other parts of device 100, may be implemented with wired or wireless communication technologies (e.g., using various known networking or computer connectivity cables, analog plugs, local wireless network, WiFi, near field communication (NFC), Bluetooth®, ANT™, ultra-wideband (UWB), etc.).

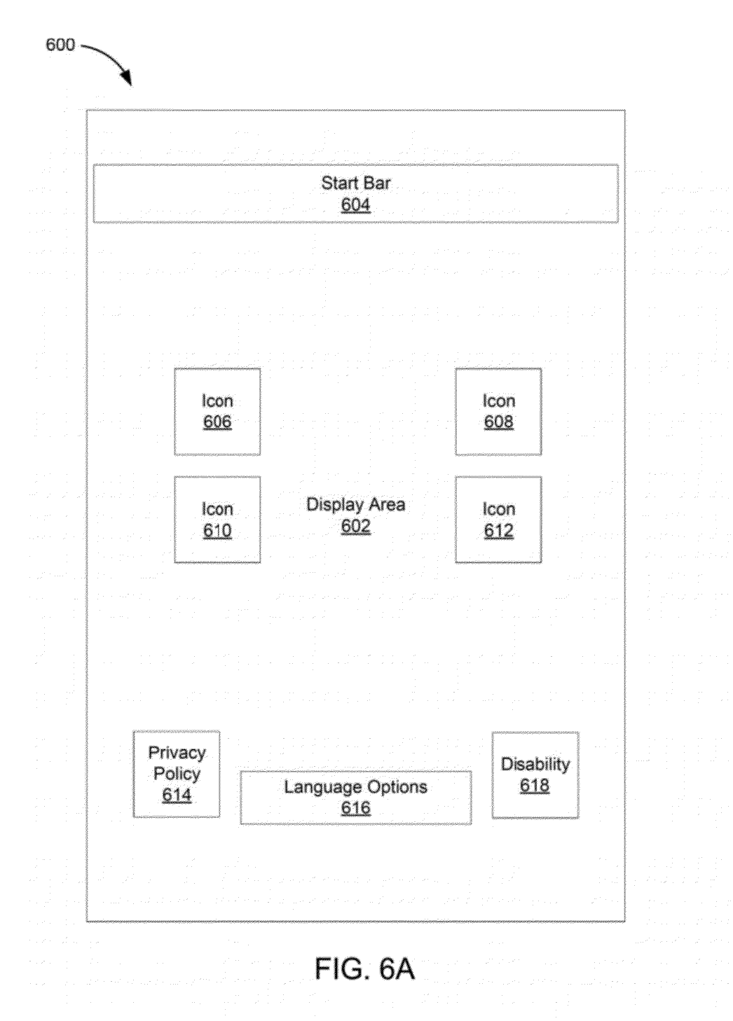

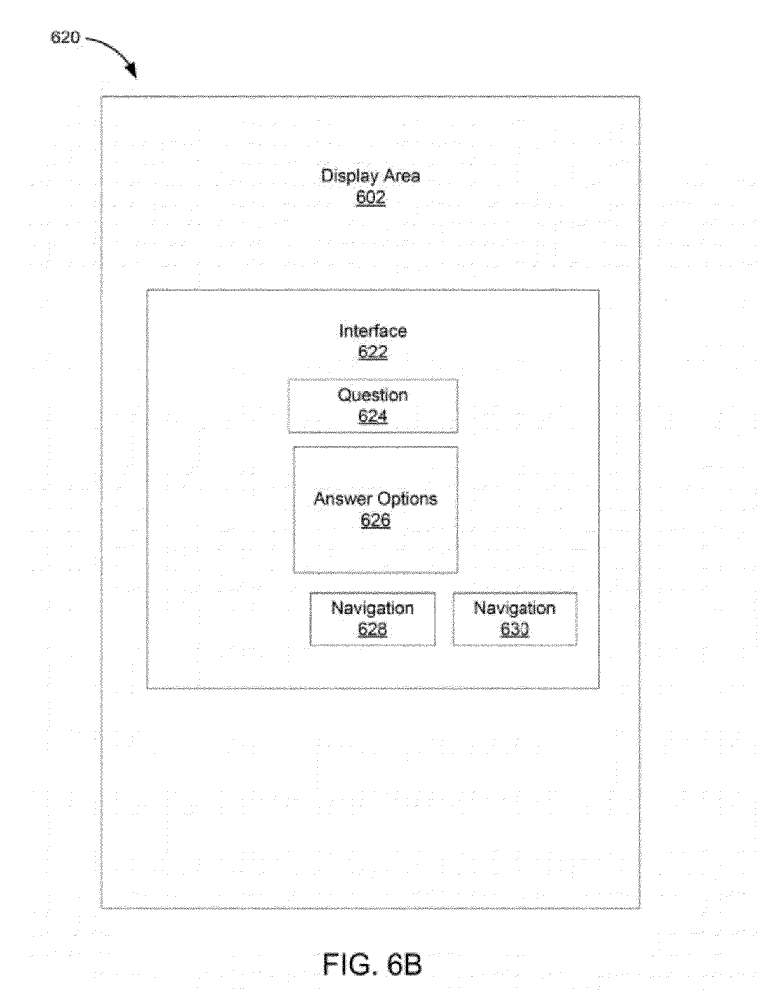

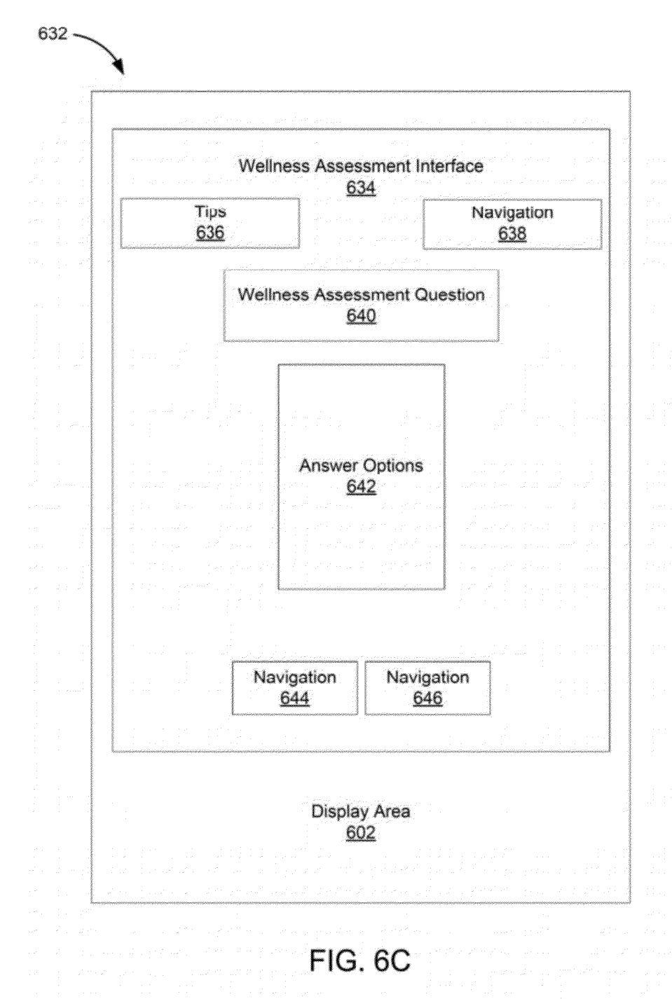

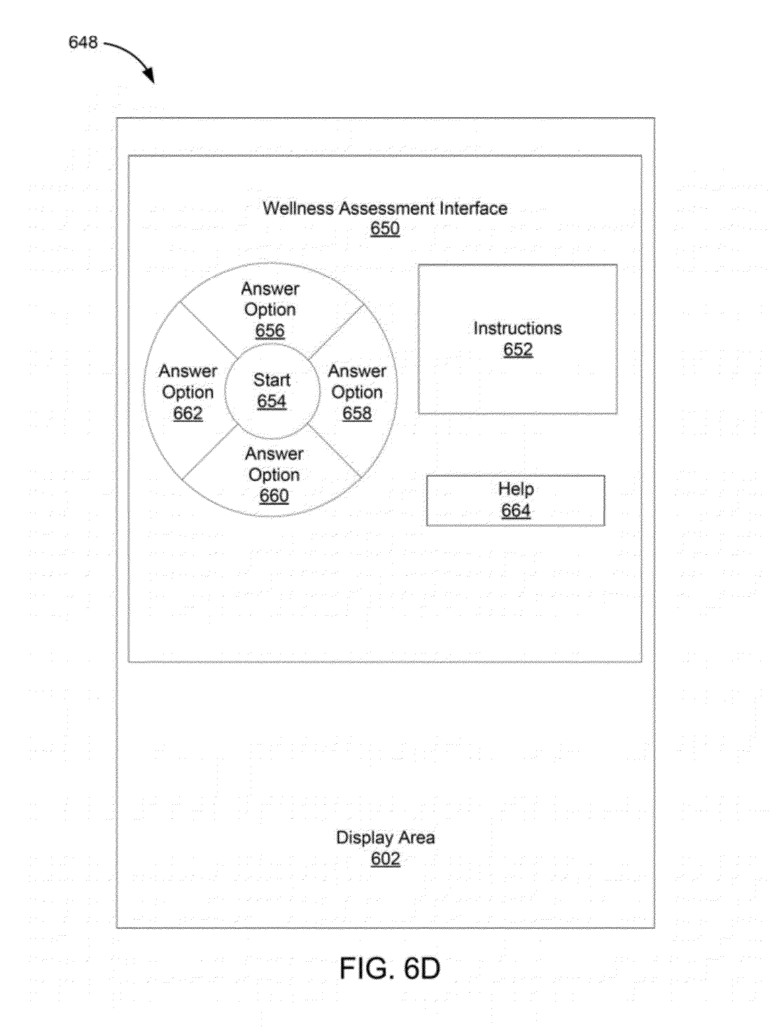

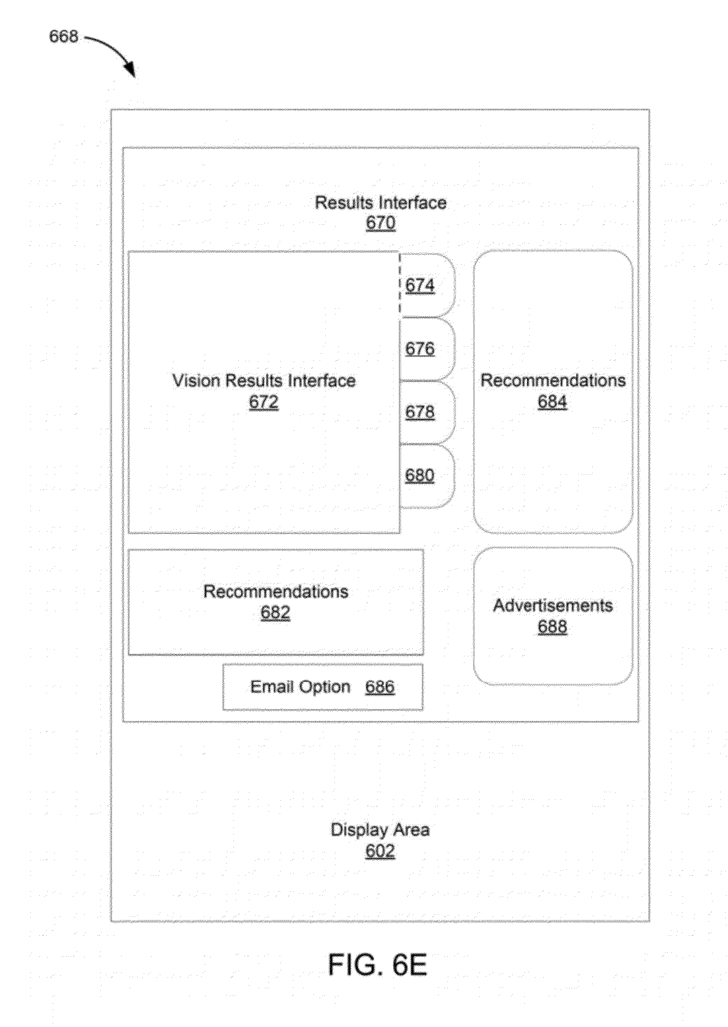

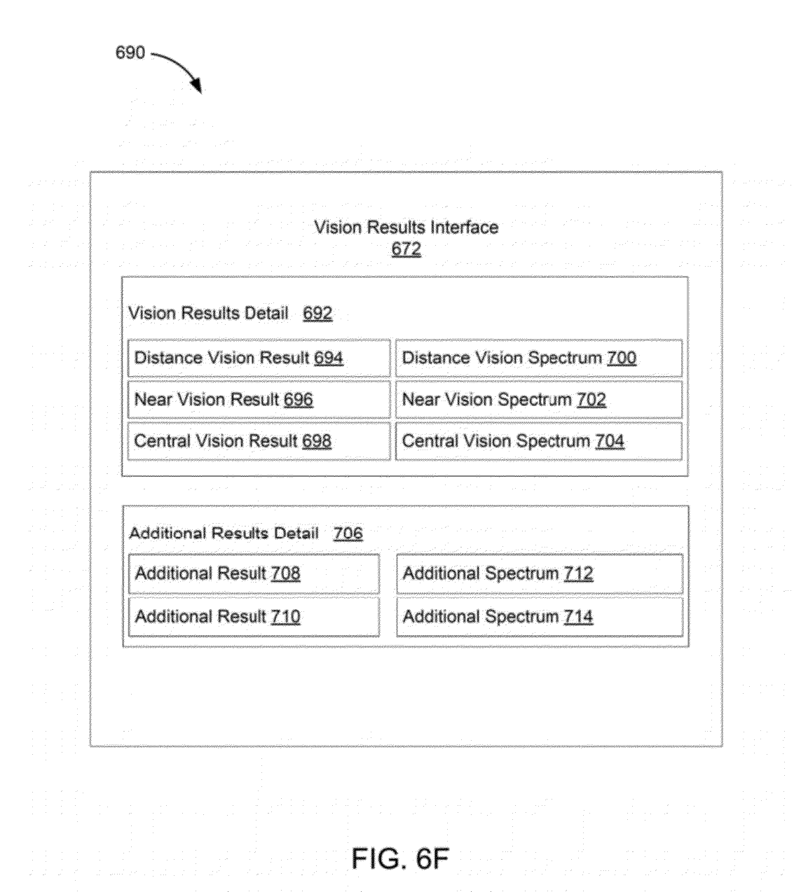

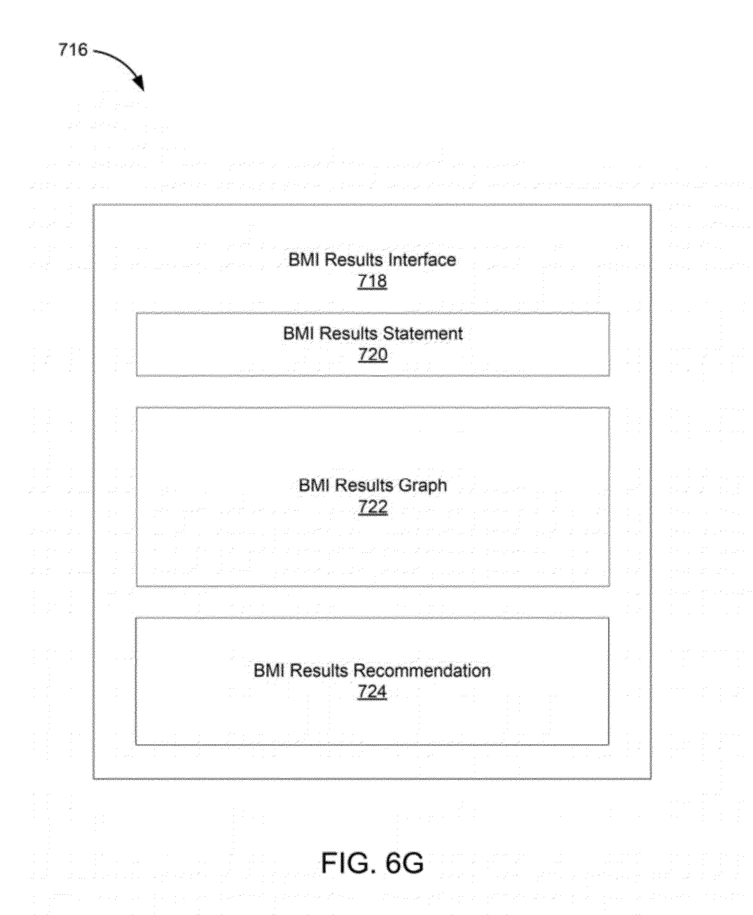

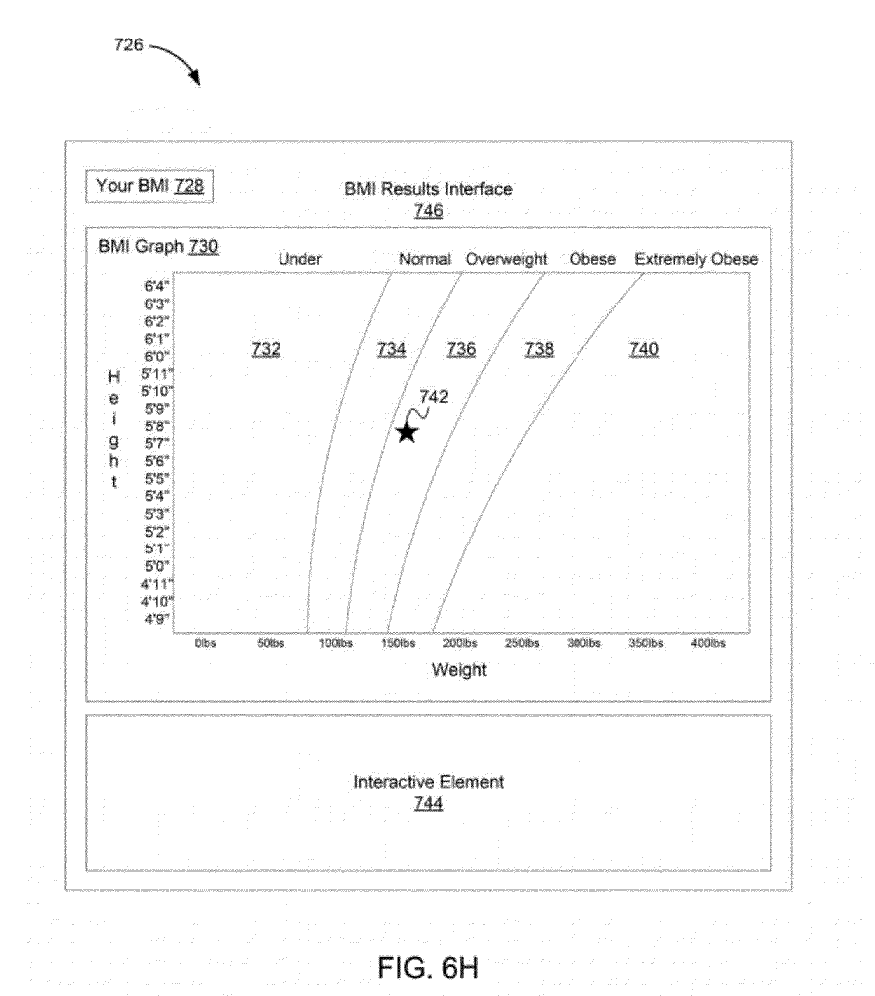

For example, display 116 may be configured to provide a user interface associated with a wellness assessment, result, recommendation, advertisement or incentive (e.g., coupons, discounts, etc.). In some examples, a user may view a user interface displayed on display 116, and interact with (e.g., select one or more options presented by the user interface) using touchscreen 104 (e.g., by touching, with a finger or a stylus, at or near a location on touchscreen 104 corresponding to the location of an option presented on display 116). In some examples, the user interface shown on display 116 may correspond with images or graphics being displayed by monitors 108 and 110, or through view portal 112. For example, various images or graphics associated with distance vision acuity assessments (e.g., comprising a Landolt C or Landolt C chart, an E Chart, a Snellen chart, etc.) may be shown through view portal 112, while the user interface provided by display 116 requests that the user make a selection associated with the distance vision acuity assessment. In this example, the user may make a selection using touchscreen 104. In other examples, monitor 110 may display images or graphics associated with near or central vision acuity assessments (e.g., Amsler grid, etc.), which may correspond with a user interface provided by display 116. Exemplary user interfaces are described in more detail herein with reference to FIGS. 6A-6H.

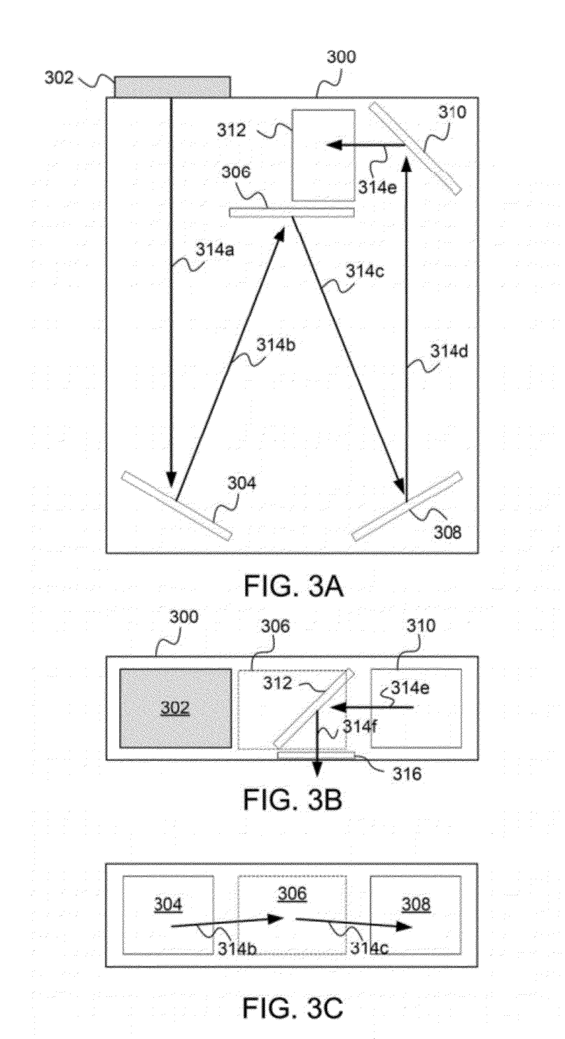

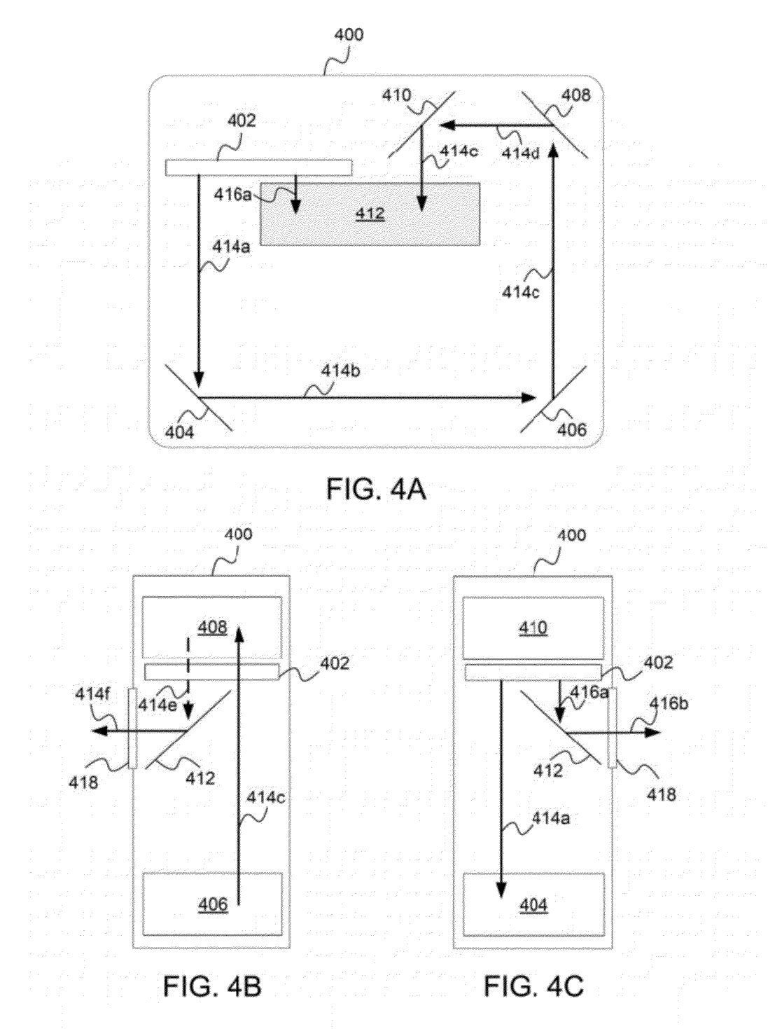

In some examples, chamber 106 may comprise a sealed optical chamber comprising a plurality of mirrors configured to reflect an image or graphic (e.g., displayed by monitor 108) along an optical path that leads through view portal 112 to a user's eye. As used herein, an “optical path” refers to the path that light takes in traveling from a monitor (e.g., monitor 108, monitor 302 in FIGS. 3A-3B, or monitor 402 in FIGS. 4A-4C) through a system of mirrors (e.g., the mirrors in FIGS. 3A-3C and FIGS. 4A-4C). Exemplary optical paths are described in more detail herein with reference to FIGS. 3A-3C and 4A-4C. In some examples, chamber 106 may be sealed to minimize or eliminate elements (e.g., dust, moisture, etc.) that may disrupt or interfere with the optical path, or otherwise diminish the quality of the light projected from monitor 108. In an example, chamber 106 may be blown out or otherwise cleaned prior to sealing. In some examples, chamber 106 may include view portal 112, which may comprise a clear glass portal through which a user may see through, for example without any distortion of the image or graphic being reflected or projected from within chamber 106. In some examples, chamber 106 may be coupled to rails 120, which may serve to guide chamber 106 as it is moved up or down using lift 114. Lift 114 may be implemented using any mechanism capable of raising or lowering any part of, or within, device 100 (e.g., chamber 106, monitors 108 and 110, display 116, etc.). In some examples, lift 114 may operate using fluid (e.g., hydraulic), pneumatic, or electro-mechanical lift devices. A person of ordinary skill in the art will appreciate that there are many different ways to implement lift 114. In other examples, displays, monitors, optical chambers, and other parts of a self-screening wellness assessment and recommendation system may be moved manually (e.g., without a motor) or semi-manually (e.g., where the movement is counterbalanced or dampened), for example, along rails or other guiding structures. In some examples, monitors 108 and 110, and display 116, may be coupled to, or affixed to, chamber 106, such that these components may move up or down along rails 120 in conjunction with chamber 106. In some examples, lift 114 may be implemented with a brake or brake assembly (not shown) to adjust (e.g., slow down) the movement of chamber 106, as well as the movement of other parts of device 100 being moved by lift 114. For example, lift 114 may use a brake to slow down or stop the movement of chamber 106, monitors 108 and 110, and display 116, when adjusting to a user's eye level (i.e., the height of a user's eyes).

In some examples, touchscreen 104 may be configured to adjust for changes in an active area of a user interface or user interface objects displayed, for example, by display 116. For example, display 116 (e.g., presenting user interface objects (e.g., buttons, checkboxes, links, icons, etc.)) may move (e.g., up or down) relative to touchscreen 104 such that the actual active area of the user interface or user interface objects is no longer the same as the user's perceived active area. In some examples, touchscreen 104 and display 116 may be implemented to translate the actual active area of such moved objects by using secondary objects (e.g., shadow buttons), which correspond to the user interface objects and are generated with coordinates to account for the new location of display 116. For example, when touchscreen 104 overlays display 116, touchscreen 104 is calibrated such that for any X, Y point on touchscreen 104, there is a point on display 116 immediately underlying that X, Y point and having a corresponding mathematical value x, y. In an example, a user interface object may be defined in a default position by a rectangle with display coordinates (x1, y1, x2, y2), corresponding to touchscreen coordinates (X1, Y1, X2, Y2). When display 116 is moved +100 pixels along the y-axis, relative to touchscreen 104, a secondary object having the same area and function as the user interface object is generated with display coordinates (x1, y1+100, x2, y2+100). In this example, when a user touches touchscreen 104 at coordinates (X1, Y1+100, X2, Y2+100), they will select the secondary object, and thereby trigger the event they intend to trigger in selecting the visible user interface object. In other examples, touchscreen 104 and display 116 may be implemented to translate the actual active area of a moved user interface or user interface objects differently.

In some examples, computer 118 may be implemented along with monitors 108 and 110, chamber 106, display 116 and touchscreen 104 to interact with a user to perform a wellness assessment and provide recommendations, advertisements, or incentives, associated with the wellness assessment. In some examples, the wellness assessment may include a vision acuity assessment (including distance, near and central vision assessment), a body mass index (BMI) assessment, a diabetes assessment, and a weight management assessment, among other types of wellness assessments. In some examples, computer 118 may be configured for use with (e.g., storage and implementation of) various software programs and applications associated with the wellness assessment, including programs and applications for providing user interfaces associated with, and performing, any of the above-mentioned assessments. In some examples, the various software programs and applications also may be associated with advertisements and incentive programs (e.g., coupons or discounts, including those linked to a person's store account or to products and services provided by the store). In some examples, these software programs and applications may be updated using any of the data communications methods described herein. For example, updated content, new self-screenings, advertisements, and incentives, may be provided. In another example, existing self-screening assessments may be updated or modified (e.g., a vision acuity assessment may be adjusted to assess whether a user has 20/10 vision, or it may be adjusted to a 20/20 acuity level, and so on).

In some examples, sensor 122 may be implemented nearing the top of housing 102 to sense the top of chamber 106 and/or monitor 108 and hence signal the arrival of chamber 106 and/or monitor 108 at the top of housing 102. In some examples, sensor 122 may communicate with lift 114, either directly or indirectly, to slow or stop the movement of chamber 106 and/or monitor 108. In other examples, device 100 may be implemented with other sensors (not shown). In some examples, these other sensors may be configured to emit a signal beam, such as an acoustic or infrared beam, and to sense (e.g., using a detector, etc.) whether the signal has been returned. For example, device 100 may use such a sensor to sense the top of a user's head. In another example, device 100 may use such a sensor to sense a distance between a user and device 100. In some examples, one or more of such sensors may communicate with computer 118, or other parts of device 100, to move view portal 112, monitor 110 and/or display 116 to a user's eye level. In other examples, input from one or more of such sensors may be processed to provide instructions (e.g., associated with a wellness assessment) to a user, for example, using a user interface displayed on display 116.

In some examples, device 100 also may be implemented with one or more speakers (not shown), a microphone (not shown), or a card reader (not shown). As used herein, a card reader refers to any data input device configured to read data from a card-shaped storage medium (e.g., identification card, credit card, store account card, etc.). In some examples, the one or more speakers may be used to provide audio instructions or help. For example, the speakers may be used to instruct the user, ask questions of the user, provide feedback to user inputs, present and explain self-screening results, present recommendations and advertisements, or provide other information associated with a wellness assessment. In some examples the microphone may be used to receive audio input. In other examples, device 100 may be implemented differently than described herein, for example with more or fewer components

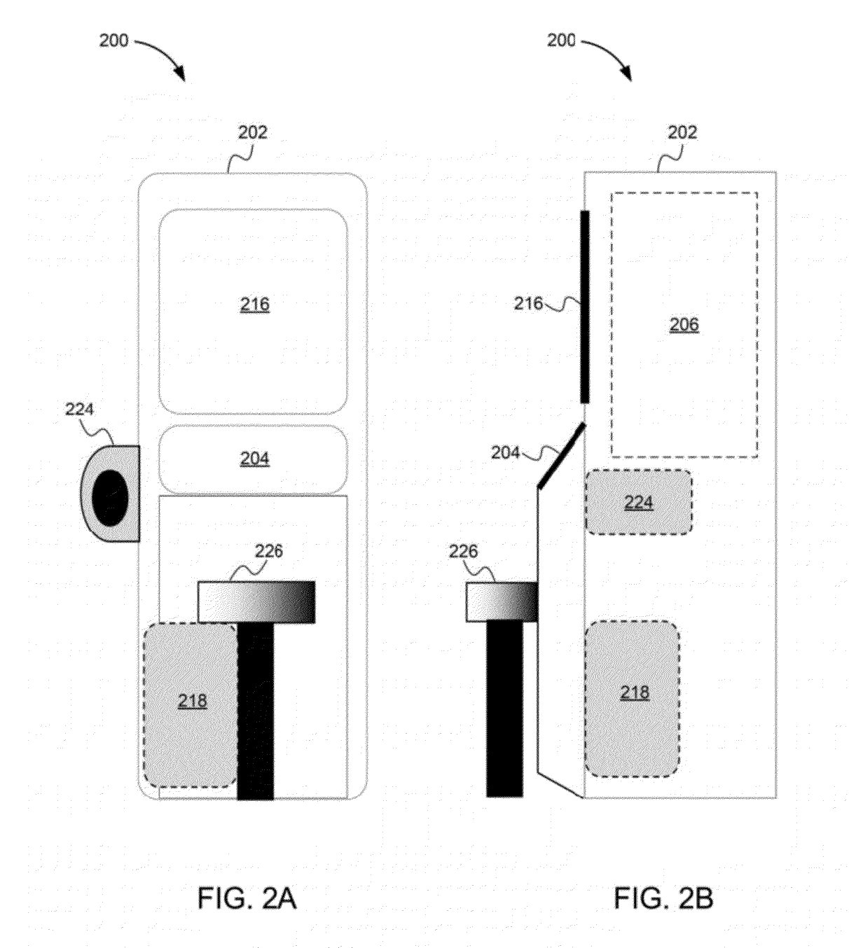

FIGS. 2A-2B illustrate front and side views, respectively, of an alternative exemplary device for implementing a self-screening wellness assessment and recommendation system. FIG. 2A shows a front view, and FIG. 2B shows a side view, of device 200. Like-numbered and named elements in these views may describe the same or substantially similar elements. In some examples, device 200 may comprise housing 202, touchscreen monitor 204, chamber 206, viewing area 216, computer 218, blood pressure assessment cuff 224, and seat 226. In some examples, touchscreen monitor 204 may be implemented in the same, or similar, manner as touchscreen 104 and display 116 in FIG. 1 (described above). In some examples, computer 218 may be implemented in the same, or similar, manner as computer 118 in FIG. 1 (described above).

In some examples, chamber 206 may be implemented in the same, or similar, manner as chamber 106 in FIG. 1 (described above). For example, chamber 206 comprise a sealed optical chamber comprising a plurality of mirrors configured to reflect an image or graphic along an optical path. Chamber 206 may be sealed to minimize or eliminate elements (e.g., dust, moisture, etc.) that may disrupt the optical path. In some examples, chamber 206 may comprise a view portal (not shown) through which the image or graphic may be projected for assessing distance vision. Images and graphics associated with distance vision assessments are described in more detail herein with reference to FIGS. 3A-3C and FIGS. 4A-4C. A user may view through viewing area 216 the graphics or symbols being projected through the view portal, as well as images or graphics being shown by one or more monitors (not shown) behind viewing area 216. In some examples, such monitors may be configured to display images or graphics for assessing near and/or central vision. In other examples, such monitors may be configured to display advertisements or other information.

In some examples, seat 226 may be configured to adjust up or down, either manually or automatically. In other examples, seat 226 also may be implemented as a scale, operable to determine a user's weight upon the user sitting on seat 226. In some examples, blood pressure assessment cuff 224 may be configured to measure a user's blood pressure. In some examples, the data gathered by blood pressure assessment cuff 224, touchscreen monitor 204, and seat 226, may be used (e.g., by computer 218) in performing a wellness assessment. In still other examples, device 200 may be implemented differently than described herein, for example with more or fewer components.

FIGS. 3A-3C illustrate front, top and bottom views, respectively, of an exemplary optical chamber in an exemplary device for implementing a self-screening wellness assessment and recommendation system. As shown, optical chamber 300 may comprise mirrors 304-312, optical path 314a-314f and view portal 316. In some examples, optical chamber 300 further may comprise, or be implemented with, monitor 302. In some examples, mirrors 304-312 may comprise any reflective surface operable to reflect light in a manner that preserves, or substantially preserves, the quality of the light prior to its contact with the mirror. In some examples, images or graphics (e.g., alpha or numeric symbols) may be projected by monitor 302 along optical path 314a to mirror 304. In some examples, mirrors 304-312 may be configured to reflect the images or graphics projected from monitor 302 along optical path 314a-314f, and ultimately through view portal 316 where the images or graphics may be viewed by a user.

In some examples, the images or graphics projected by monitor 302 may be associated with a distance vision assessment. For example, the image or graphic may comprise a Landolt C, or Landolt C chart, an E Chart, a Snellen chart, or other graphic or image used in distance vision acuity assessments. In some examples, the size of the image or graphic being displayed may vary depending on the vision acuity standard (e.g., 20/10, 20/20, 20/40, etc.) being used in the assessment. The size of the image or graphic, as well as the vision acuity standard being used in the assessment, may be adjusted using software or firmware implemented by monitor 302, or by another computing device communicating data with monitor 302. In other examples, the size of the image or graphic also may vary depending on the total distance of optical path 314a-314f (e.g., 10 feet, 15 feet, 20 feet, etc.). A person of ordinary skill in the art will comprehend the possible variations in the total distance of optical path 314a-3141, and how to adjust the size of the image or graphic to assess vision at a given acuity standard. In still other examples, optical chamber 300 may be implemented differently than described herein, for example with more or fewer components.

FIGS. 4A-4C illustrate front, right side and left side views, respectively, of an alternative exemplary optical chamber in an exemplary device for implementing a self-screening wellness assessment and recommendation system. As shown, optical chamber 400 may comprise monitor 402, mirrors 404-412, optical path 414a-414f, optical path 416a-416b, and view portal 418. In some examples, mirrors 404-412 may be implemented in the same, or similar, manner as mirrors 304-312 in FIGS. 3A-3C (described above). In some examples, mirrors 404-412 may be configured to reflect images or graphics projected from monitor 402 along optical path 414a-414f or optical path 416a-416b. For example, monitor 402 may project an image or graphic associated with a distance vision acuity assessment along optical path 414a-414f, reflected by mirrors 404-412, respectively, and through view portal 418 where the image or graphic may be viewed by a user. In another example, monitor 402 may project an image or graphic associated with a near or central vision acuity assessment along optical path 416a-416b, reflected by mirror 412 and through view portal 418.

In some examples, monitor 402, mirror 412 and view portal 418 may be configured to move vertically to accommodate a user's eye level. In some examples, monitor 402, mirror 412 and view portal 418 may be coupled together such that they may move as a unit. As monitor 402, mirror 412 and view portal 418 move down together, optical path 414a shortens in distance and optical path 414e lengthens with a corresponding distance. In this example, the total distance of optical path 414a-414f remains constant. Also in this example, the total distance of optical path 416a-416b also remains constant. In some examples, the vertical movement may be carried out using a lift (not shown). In other examples, optical chamber 400 may be implemented differently than described herein, for example with more or fewer components.

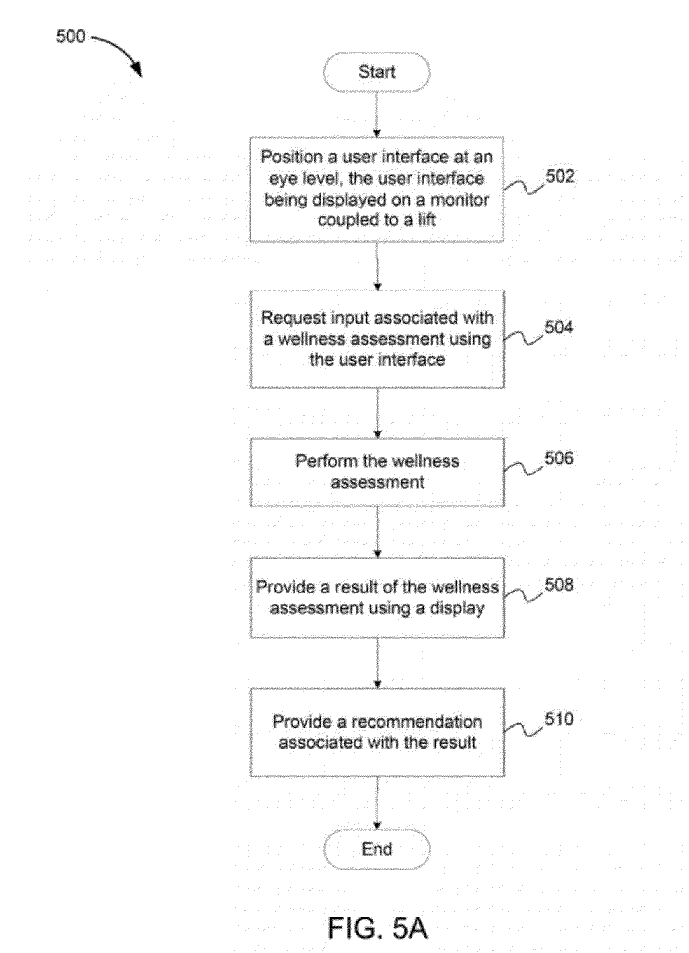

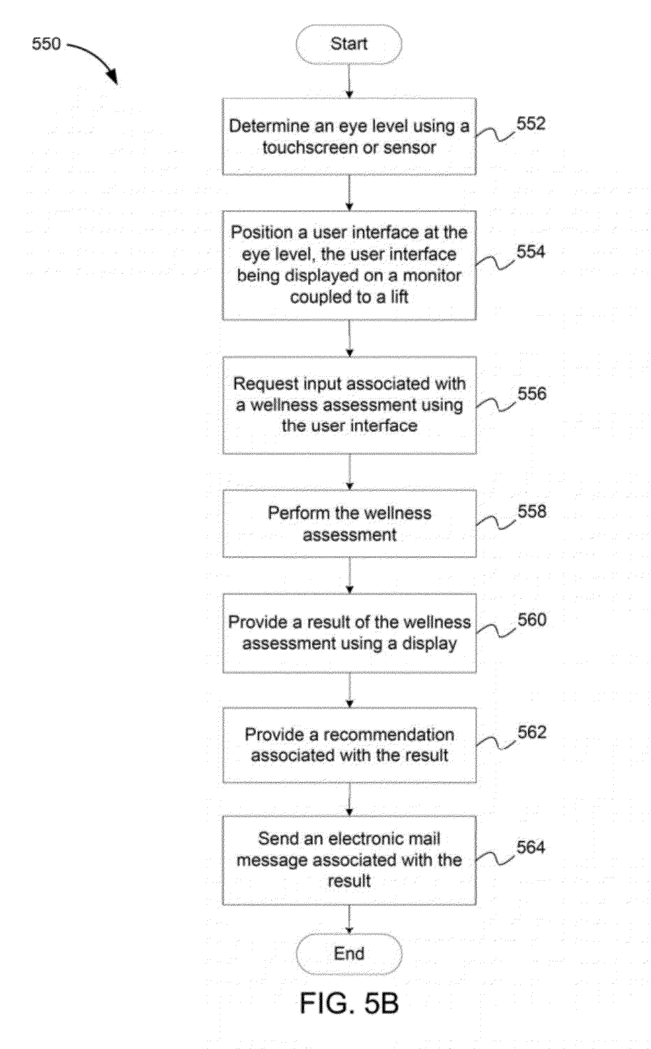

FIGS. 5A-5B illustrate exemplary processes for providing a self-screening wellness assessment and recommendation. In some examples, process 500 may begin with positioning a user interface at an eye level, the user interface being displayed on a monitor coupled to a lift (502). For example, a user may indicate, or specify, the user's eye level by touching a touchscreen or touchscreen monitor (e.g., touchscreen 104, touchscreen monitor 204, etc.) at that height. In another example, a sensor may determine the top of a user's head and estimate eye level using that data. Once an eye level is determined, the lift may move the monitor so that the user interface is being displayed at the user's eye level. In some examples, the placement of a user interface may be adjusted multiple times in order to present various parts of the user interface at the eye level. In another example, a user interface may be moved out of the way (e.g., down) so that a view portal or another monitor for viewing graphics associated with a vision acuity assessment may be placed at a user's eye level. In this example, the view portal, monitors and/or user interface may be adjusted up and down multiple times to make the assessment process more comfortable for the user.

In some examples, once the user interface is positioned at a user's eye level, input associated with a wellness assessment may be requested using the user interface (504). In some examples, this input being requested may include the user's age, sex, race or ethnicity, height, weight, waist size, smoking and drinking habits, responses to medical history questions, responses to vision acuity assessment questions, responses to other assessment questions, or any other information that may be relevant to assessing a user's wellness. In some examples, a user may provide this input in response to questions or instructions provided by a user interface, and may do so by making selections using a touchscreen or touchscreen monitor.

In some examples, while, or after, the requested input is being provided by the user, the wellness assessment may be performed (506). In some examples, the wellness assessment may be performed using a computer, or other computing device, and various software-implemented algorithms. In some examples, the requested input (e.g., user's age, sex, race or ethnicity, height, weight, waist size, blood pressure, activity level (e.g., amount of exercise), smoking and drinking habits, responses to medical history questions, responses to vision acuity assessment questions, other assessment questions, etc.) may be used to assess various aspects of a user's wellness, including their vision acuity, eye health (e.g., risk or level of cataracts, dry eyes, ocular allergies, glaucoma, macular degeneration, ocular sun (UV) damage, etc.), risks for various diseases or illnesses (e.g., diabetes, some types of cancer, sleep apnea, osteoarthritis, cardiovascular diseases, liver or gallbladder diseases, etc.). For example, part of the wellness assessment may include a determination of the user's BMI, which may be calculated using requested input associated with a user's weight and height. In some examples, the requested weight and height input may be determined automatically (e.g., using one or more sensors and/or scales, as described herein) or by asking questions (e.g., relating to the user's weight and height) using a user interface and receiving a user's responses to the questions. The user's BMI may be calculated using the formula: BMI=(Weight×703)/Height'. In this example, weight may be provided in pounds (lbs), and height in inches. In other words, a user's BMI is the user's mass, in pounds, multiplied by 703, and divided by the square of the user's height, in inches. In other examples, a BMI calculation may be refined by information associated with a user's age and gender.

After the wellness assessment is performed, a result of the wellness assessment may be provided using a display (508). In some examples, the result may include vision acuity screening results, including whether or not a user answered correctly in response to assessment questions related to distance vision, near vision or central vision. In another example, the result may include other health or wellness screening results (e.g., a user's BMI, diabetes risk, dry eye level or risk, ocular allergies level or risk, sun damage level or risk, flu risk, etc.). In some examples, the result may comprise a graphic representation of a spectrum (e.g., good to bad, correctly answered to not okay, etc.) and an indication of where on the spectrum the user's vision acuity falls. In other examples, the result may comprise a graphic representation of a spectrum (e.g., mild to moderate to severe). In still other examples, the result also may comprise an interactive graph. For example, a result associated with a user's BMI may include a statement of the user's BMI, information about whether the user's BMI falls within a particular category (e.g., under, normal, overweight, obese, etc.), a graph showing ranges of weight and height along the X- and Y-axes, and areas of the graph corresponding to various categories (e.g., underweight, normal weight, overweight, obese, etc.), with an indication (e.g., by an icon, dot, cross-hair graphic, etc.) of the user's weight and height on the graph (FIG. 6H), as well as an interactive element (e.g., where the user may see how they may move between the weight or BMI categories according to various options (e.g., by losing 5 lbs, by losing 25 lbs, by gaining 10 lbs, etc.).

In some examples, a recommendation associated with the result also may be provided (510). In some examples, the recommendation may include suggestions for lowering the user's risks for certain diseases, or mitigating risk factors for those diseases, may be provided using the user interface. For example, if a user's BMI indicates they are in the overweight or obese categories, the recommendation may include suggestions for losing weight, and other measures to reduce the user's risk of diabetes, coronary heart disease, hypertension, high cholesterol, and various types of cancers associated with a higher BMI. In other examples, process 500 may be implemented differently than described herein, for example with more or fewer steps.

FIG. 5B illustrates an alternative process for providing a self-screening wellness assessment and recommendation. In some examples, process 550 may begin with determining an eye level using a touchscreen or sensor (552). As described in more detail above, a sensor may be employed to automatically determine a user's eye level, or a user may simply touch a touchscreen at their eye level. Once an eye level is determined, a user interface may then be positioned at the eye level, the user interface being displayed on a monitor coupled to a lift (554). In some examples, input associated with a wellness assessment may be requested using the user interface (556). In some examples, while the input is being received, or afterward, the wellness assessment may be performed (e.g., using a computer or other computing device) (558). After the wellness assessment is performed, a result of the wellness assessment may be provided using a display (560). In some examples, a recommendation associated with the result also may be provided, using the display or other means (562). For example, an electronic mail message associated with the result may be sent (564), which may include the recommendation. In other examples, results and recommendations associated with a user's wellness assessment may be provided in other manners as well, for example, using a text message, a web application, a mobile application, or other means by which a user may wish to access information associated with their health and wellness. In still other examples, process 550 may be implemented differently than described herein, for example with more or fewer steps.

FIGS. 6A-6H illustrate wireframes of exemplary interfaces associated with a self-screening wellness assessment and recommendation system. FIG. 6A illustrates a wireframe of an exemplary wellness assessment starting interface on a display. As shown, wireframe 600 comprises display area 602, start bar 604, icons 606-612, privacy policy 614, language options 616 and disability feature 618. In some examples, display area 602 may correspond to the display area of a display (e.g., monitor, screen, etc., as described herein). In some examples, start bar 604 may be implemented as a button, link, or other navigation means (hereafter collectively referred to as “buttons”), for bringing up a starting screen or interface (e.g., interface 622 or wellness assessment interface 634, as shown in FIGS. 6B and 6C, respectively) for beginning a wellness assessment. In other examples, start bar 604 may instruct a user to touch display area 602 at a height corresponding to the user's eye level, for example, to determine an eye level for adjusting the height of a user interface. In still other examples, start bar 604 may display an instruction to “touch here to start screening.”

In some examples, icons 606-612 may show icons associated with various health or wellness topics. In some examples, icons 606-612 may be implemented as buttons enabling a user to navigate to screens or interfaces associated with the health or wellness topic represented by the icon. Some examples of topics include contact lenses, allergies, glasses, weight loss, exercise, and others. For example, icon 606 may show an icon associated with eye health (e.g., a person wearing glasses, a pair of glasses, etc.), and selecting icon 606 may enable a user to navigate to a screen providing information, and other links and features associated with eye health. In other examples, icons 606-612 may be associated with advertisements for products. For example, the self-screening wellness assessment and recommendation system may be located in or near a store providing health and wellness products or categories of products, for which selecting one or more of icons 606-612 may pull up more information. In some examples, language options 616 may be implemented using one or more buttons (e.g., labeled with various language options (e.g., English, Spanish, Chinese, etc.)). In an example, selection of a language option provided by language options 616 may change the language used on the current screen being displayed, and all subsequent screens being displayed until a user session ends, or a subsequent language selection is made. In some examples, a privacy policy may be implemented in association with the self-screening wellness assessment and recommendation system, which may be accessed by selecting privacy policy 614. For example, a user may pull up a screen (e.g., pop-up, embedded, replacement, etc.) displaying the text of the privacy policy by selecting privacy policy 614. In some examples, disability feature 618 may enable a user to access tools for use by disabled users (e.g., to lower a display of a user interface to a wheelchair level, to enable audio capabilities, etc.). In other examples, wireframe 600 may be implemented differently than described herein, for example with more or fewer features.

FIG. 6B illustrates a wireframe of an exemplary interface for requesting input from a user associated with a self-screening wellness assessment. As shown, wireframe 620 comprises display area 602 and interface 622, which in turn presents question 624, answer options 626, and navigations 628 and 630. In some examples, interface 622 may be implemented as a user interface configured to request information from a user. In some examples, interface 622 may do so in a question and answer format. In some examples, answer options 626 may present answer options in the form of multiple choice options, keypad entry, keyboard entry, or other data entry options. For example, question 624 may show a question requiring a “yes” or “no” answer, such as “Do you get an annual influenza (or flu) vaccination?”. In this example, answer options 626 may present two buttons, one labeled “Yes” and one labeled “No.” In another example, question 624 may ask a user “What is your gender?”. In this example, answer options 626 may present two buttons, one labeled “Female” and one labeled “Male.” In yet another example, question 624 may ask a user “What is your height?”. In this example, answer options 626 may present a numeric keypad with fields indicating number selections (e.g., for feet, inches, meters, etc.). In other examples, question 624 may ask a question wherein a user may select more than one answer option (e.g., “Please indicate your vision correction methods, if any.”), and answer options 626 would allow the user to select one, or more than one, answer option before continuing. In some examples, navigation 628 and 630 may be implemented as buttons enabling a user to navigate back and forth among various screens associated with the user interface. For example, navigation 628 and 630 may be labeled “Continue,” “Back,” “Start Over,” or other label indicating where the navigation button will lead. In other examples, answer options 626 may present answer buttons that themselves serve as navigation buttons. For example, where the buttons presented in answer options 626 present an either/or condition, or where the user is required to select I 0 an exclusive answer option (e.g., cannot select more than one answer), the selection of an answer may advance the user to the next screen automatically. In other examples, wireframe 620 may be implemented differently than described herein, for example with more or fewer features.

FIG. 6C illustrates a wireframe of an exemplary wellness assessment interface for requesting input from a user associated with a self-screening wellness assessment. As shown, wireframe 632 comprises display area 602 and wellness assessment interface 634, which in turn presents tips 636, navigation 638, wellness assessment question 640, answer options 642, navigation 644 and 646. In some examples, wellness assessment question 640 and answer options 642 may be implemented in the same, or similar, manner as question 624 and answer options 626 in FIG. 6B. In some examples, navigation 638, 644 and 646 may be implemented in the same, or similar, manner as navigation 628 and 630 in FIG. 6B. In some examples, tips 636 may display useful information associated with answering wellness assessment question 640. In other examples, tips 636 may be implemented as a button enabling a user to navigate to another screen with tips, or other information, associated with the wellness assessment or interacting with the user interface. In still other examples, wireframe 632 may be implemented differently than described herein, for example with more or fewer features.

FIG. 6D illustrates a wireframe of an exemplary wellness assessment interface for requesting input from a user associated with a self-screening wellness assessment. As shown, wireframe 648 comprises display area 602 and wellness assessment interface 650, which in turn presents instructions 652, start 654, answer options 656-662 and help 664. In some examples, wellness assessment interface 650 may be associated with a vision acuity assessment. In some examples, instructions associated with the vision acuity assessment may be presented by instructions 652. For example, instructions 652 may present text or graphics instructing a user to select (e.g., by pressing or touching) start 654 to begin the assessment, and then to look into a view portal or at a monitor to view an image or graphic associated with the assessment. In an example, the image or graphic may be a Landolt C, or Landolt C chart, an E Chart, a Snellen chart, or other symbol or chart. For example, if an image of an upside down letter (e.g., an E or Landolt C ring) is shown on a monitor or through a view portal, answer options 656-662 may present variations of the letter, with one of the variations being the correct upside down letter. In another example, the image or graphic may be an Amsler grid, for central vision screening. An Amsler grid may be comprised of horizontal and vertical lines with a dot in the center of the grid. A user may be instructed using instructions 652 to look at the Amsler grid, with one eye, and then the other, and to indicate using answer options 656-662 whether the user is able to see all four corners of the grid, the dot positioned in the center of the grid, distortions or waves in any of the lines, or provide other input associated with a central vision assessment. In some examples, help 664 may display helpful information associated with wellness assessment interface 650. In other examples, help 664 may be implemented as a button enabling a user to navigate to another screen with additional information (e.g., a frequently asked questions (FAQ) and answers screen) associated with the wellness assessment being performed. In still other examples, wireframe 648 may be implemented differently than described herein, for example with more or fewer features.

FIG. 6E illustrates a wireframe of an exemplary results interface for displaying results associated with a self-screening wellness assessment. As shown, wireframe 668 comprises display area 602 and results interface 670, which in turn presents vision results interface 672, tabs 674-680, recommendations 682 and 684, email option 686 and advertisements 688. In some examples, results interface 670 may be configured to present an interactive user interface for presenting the results of a wellness assessment. In some examples, results interface 670 may comprise sub-components, including other interfaces, such as vision results interface 672. A user may use tabs 674-680 to navigate among the various other interfaces. For example, tab 674 (shown as selected) may be labeled “vision,” and correspond with vision results interface 672 (see FIG. 6F). When tab 674 is selected, vision results interface 672 is visible and presents the results of a user's vision assessment. In another example, tabs 676-680 may be labeled “BMI,” “diabetes,” “coupons,” “other info,” or may be otherwise labeled to correspond to the a sub-component of results interface 670. In this example, tab 676 may be labeled BMI, and thus correspond with a BMI results interface (not shown in FIG. 6E; see FIG. 6G). When tab 676 is selected, a BMI results interface is displayed in place of vision results interface 672. In other examples, a results interface may be implemented with more or fewer tabs and sub-component interfaces, depending on a user's responses to assessment questions. For example, if a user indicates they are a smoker, an additional tab and sub-component interface (not shown) may be included in wireframe 668 to address issues, risks and information associated with smoking.

In some examples, recommendations 682 and 684 may display various recommendations associated with the wellness assessment. For example, recommendations 682 may display actions a user can take to mitigate the conditions, illnesses or diseases for which they may be at risk (e.g., get an annual flu vaccination, make an appointment with an eye care professional, lose or gain weight, etc.). In another example, recommendations 684 may display other types of recommendations, including information listings of service providers (e.g., doctors, ophthalmologists, optometrists, medical specialists, etc.), stores, or products that may help with any issues identified in the wellness assessment. In some examples, such listings may include information such as the name, specialty, and location of the service provider.

In some examples, advertisements 688 may display advertisements. These advertisements may be associated with the wellness assessment as a whole, or they may be tailored to the results of, or recommendations for, the wellness assessment of an user. For example, advertisements 688 may be implemented with icons, such as the icons described in FIG. 6A. In another example, advertisements 688 may display one or more advertisements, for example, associated with in-store products that may be helpful to the user (e.g., a flu shot for a user that indicates the user has not obtained a flu shot, sunglasses for a user that presents with ocular sun damage, etc.). In some examples, advertisements 688 may be configured to cycle through displaying two or more advertisements.

In some examples, email option 686 may be implemented as a button to enable a user to request that the results and recommendations of the user's wellness assessment be sent to the user's electronic mail (herein referred to as “email”) account in an email message. In some examples, email option 686 may be implemented as a button that enables a user to navigate to a separate screen, or causes a new interface to be overlaid on the results page, where the user may enter an email address (e.g., a virtual keyboard) to which the results and recommendations of the user's wellness assessment may be sent. In other examples, email option 686 may be implemented as a button that causes an email comprising said results and recommendations to be emailed automatically to an email account on file, or previously obtained, for the session (e.g., by swiping a store account or other identification card through a card reader). In still other examples, wireframe 668 may be implemented differently than described herein, for example with more or fewer features.

As will be appreciated by those skilled in the art, the various interfaces described in FIGS. 6B-6E also may be configured to perform, and display results and recommendations for, a diabetes risk assessment. In some examples, information associated with a user's age, sex, race/ethnicity, family medical history, height, weight, medical history (e.g., history of hypertension), and activity level (e.g., exercise level), among other information, may be elicited to assess a user's diabetes risk.

FIG. 6F illustrates a wireframe of an exemplary vision results interface for displaying vision results associated with a self-screening wellness assessment. As shown, wireframe 690 comprises vision results interface 672, which in turn presents vision results detail 692 and additional results detail 706. In some examples, vision results detail 692 may comprise distance vision result 694, near vision result 696, central vision result 698, each of which may provide detailed results associated with an assessment of a user's distance, near and central vision screenings, respectively. For example, distance vision results 694 may indicate that a user answered correctly most or all of the distance vision screening questions. In some examples, distance vision spectrum 700 may correspond with distance vision results 694, graphically presenting a user's distance vision screening results on a spectrum (e.g., between all correctly answered and none correctly answered). Near vision results 696 and near vision spectrum 702 likewise may provide details regarding how many of the near vision screening questions were answered correctly; and central vision results 698 and central vision spectrum 704 likewise may provide details regarding how many of the central vision screening questions were answered correctly. In some examples, distance vision spectrum 700, near vision spectrum 702, and central vision spectrum 704 may use several colors, each fading into the other, to correspond to ranges within the spectrum (e.g., green to indicate a good range (i.e., many or all answered correctly), yellow to indicate an intermediate range (i.e., some or many answered correctly), and red to indicate an unacceptable or bad range (i.e., many or all answered incorrectly)). In some examples, additional results detail 706 may comprise additional result 708 and 710, and additional spectrum 712 and 714. Additional result 708 and 710, and additional spectrum 712 and 714, may provide details regarding assessments of other health factors (e.g., associated with eye health), including levels or risks of dry eye, ocular allergies, sun damage, flu, and other conditions, the risks or levels for which may be assessed using the user interfaces and input techniques described herein. For example, additional result 708 and additional spectrum 712 may indicate that the user has a moderate risk or level of dry eye. In another example, additional result 710 and additional spectrum 714 may indicate that the user has a high or severe risk of sun damage. In other examples, wireframe 690 may be implemented differently than described herein, for example with more or fewer features.

FIG. 6G illustrates a wireframe of an exemplary BMI results interface for displaying BMI results associated with a self-screening wellness assessment. As shown, wireframe 716 comprises BMI results interface 718, which in turn presents BMI results statement 720, BMI results graph 722 and BMI results recommendation 724. In some examples, BMI results interface 718 may be shown as part of results interface 670 (FIG. 6E) when the BMI tab (e.g., one of tabs 676-680) is selected. In some examples, BMI results statement 720 may provide a statement of a user's BMI results. For example, BMI results statement 720 may indicate a user's BMI, a healthy weight range for the user's height, and a weight category in which the user currently falls (e.g., underweight, normal weight, overweight, obese, extremely obese, etc.). In some examples, BMI results graph 722 may indicate a user's BMI in a graph format (an example of which is shown and described further in FIG. 6H). In some examples, BMI results recommendation 724 may provide a statement or list of recommendations associated with a user's BMI. For example, if a user falls below the normal weight range for his or her height, recommendation 724 may indicate a recommendation to gain weight, including information regarding techniques for gaining weight and other conditions, illnesses, or diseases for which the user is at risk based upon the user's BMI results. In other examples, wireframe 716 may be implemented differently than described herein, for example with more or fewer features.

FIG. 6H illustrates a wireframe of an alternative exemplary BMI results interface for displaying BMI results associated with a self-screening wellness assessment. As shown, wireframe 726 comprises BMI results interface 746, which in turn presents your BMI 728, BMI graph 730 and interactive element 744. As shown, BMI graph 730 may comprise an X-axis comprised of weight values, a Y-axis comprised of height values, and graphical indication of areas 732-740 corresponding to various weight categories, including underweight, normal weight, overweight, obese and extremely obese. In some examples, BMI graph 730 further may comprise graphic 742 (e.g, a star, a cross-hair, a dot, or other symbol), for indicating a BMI value on BMI graph 730 (e.g., a user that is 5′7″ and weighs 160 lbs may slightly be overweight given other factors provided (e.g., sex, age, etc.)). Thus, in some examples, areas 732-740 and their corresponding labels may change in relation to the given X- and Y-axes based upon a user's sex, age, and other factors. For example, the area of the graph corresponding to a normal weight range may be different for a female versus a male user, a younger versus an older user, or based on other parameters.

In some examples, a user may manipulate the placement of graphic 742 through interactive element 744. In some examples, interactive element 744 may provide buttons (not shown) to enable a user to change the placement of graphic 742. For example, interactive element 744 may provide selections for a user to view where their position on BMI graph 730 may change if they lost weight or gained weight (e.g., where graphic 742 may move given a certain amount of weight lost or gained). In other examples, interactive element 744 also may provide a button that a user may select to place graphic 742 back where the user's current height and weight falls on BMI graph 730. In some examples, interactive element 744 also may provide instructions associated with such buttons, as well as other information associated with a user's BMI assessment. In other examples, wireframe 726 may be implemented differently than described herein, for example with more or fewer features.

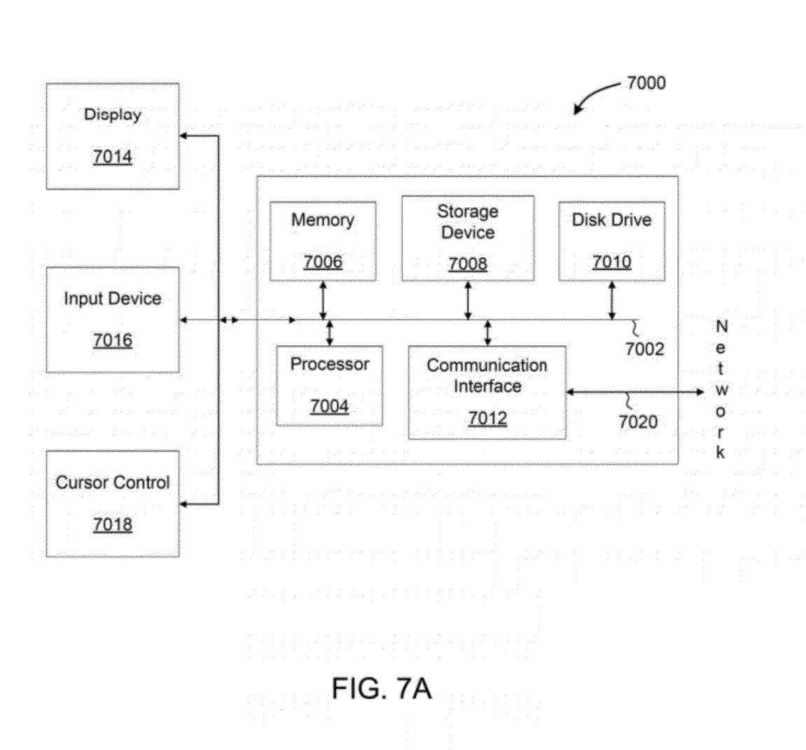

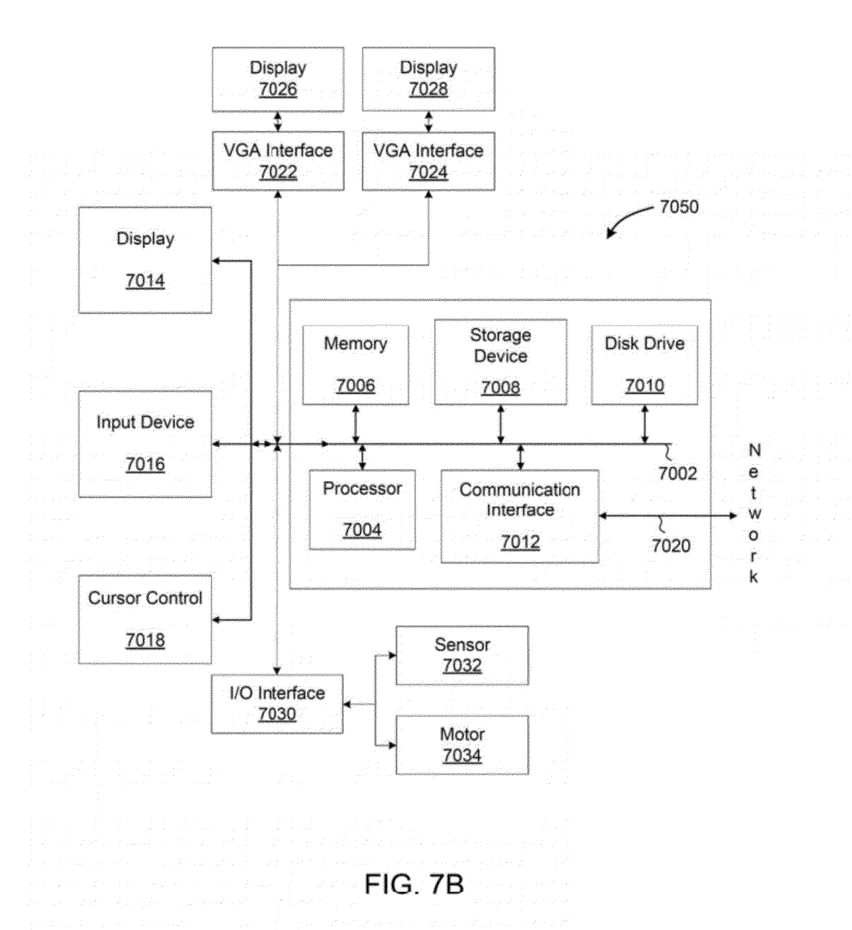

FIG. 7A illustrates an exemplary computer system suitable for implementation of a self-screening wellness assessment and recommendation system. In some examples, computer system 7000 may be used to implement computer programs, applications, methods, processes, or other software to perform the above-described techniques. Computer system 7000 includes a bus 7002 or other communication mechanism for communicating information, which interconnects subsystems and devices, such as processor 7004, system memory 7006 (e.g., RAM), storage device 7008 (e.g., ROM), disk drive 7010 (e.g., magnetic or optical), communication interface 7012 (e.g., modern, Ethernet card, wireless internet card, etc.), display 7014 (e.g., CRT, LED, LCD, plasma, OLED, etc.), input device 7016 (e.g., keyboard), and cursor control 7018 (e.g., mouse or trackball).

According to some examples, computer system 7000 performs specific operations by processor 7004 executing one or more sequences of one or more instructions stored in system memory 7006. Such instructions may be read into system memory 7006 from another computer readable medium, such as static storage device 7008 or disk drive 7010. In some examples, hard-wired circuitry may be used in place of or in combination with software instructions for implementation.

The term “computer readable medium” refers to any tangible medium that participates in providing instructions to processor 7004 for execution. Such a medium may take many forms, including but not limited to, non-volatile media and volatile media. Non-volatile media includes, for example, optical or magnetic disks, such as disk drive 7010. Volatile media includes dynamic memory, such as system memory 7006.

Common forms of computer readable media includes, for example, floppy disk, flexible disk, hard disk, magnetic tape, any other magnetic medium, CD-ROM, any other optical medium, punch cards, paper tape, any other physical medium with patterns of holes, RAM, PROM; EPROM, FLASH-EPROM, any other memory chip or cartridge, or any other medium from which a computer can read.

Instructions may further be transmitted or received using a transmission medium. The term “transmission medium” may include any tangible or intangible medium that is capable of storing, encoding or carrying instructions for execution by the machine, and includes digital or analog communications signals or other intangible medium to facilitate communication of such instructions. Transmission media includes coaxial cables, copper wire, and fiber optics, including wires that comprise bus 7002 for transmitting a computer data signal.

In some examples, execution of the sequences of instructions may be performed by a single computer system 7000. According to some examples, two or more computer systems 7000 coupled by communication link 7020 (e.g., LAN, PSTN, wireless network, etc.) may perform the sequence of instructions in coordination with one another. Computer system 7000 may transmit and receive messages, data, and instructions, including program, i.e., application code, through communication link 7020 and communication interface 7012. Received program code may be executed by processor 7004 as it is received, and/or stored in disk drive 7010, or other non-volatile storage for later execution.

FIG. 7B illustrates an alternative exemplary computer system suitable for implementation of a self-screening wellness assessment and recommendation system. As shown, computer system 7050 may include bus 7002, processor 7004, memory 7006, storage device 7008, disk drive 7010, communication interface 7012, display 7014, input device 7016, cursor control 7018, VGA interfaces 7022 and 7024, displays 7026 and 7028, I/O interface 7030 (e.g., USB, RS232, etc.), sensor 7032 and motor 7034. Like-numbered and named elements in FIGS. 7A and 7B may describe the same or substantially similar elements. In some examples, I/O interface may be implemented as a reconfigurable I/O device operable to control (e.g., handle relays with and between) sensor 7032 and motor 7034. In some examples, VGA interfaces 7022 and 7024 may be implemented to support displays 7026 and 7028, respectively.

As will be appreciated by those skilled in the art, the various embodiments of a self-screening wellness assessment and recommendation system, and associated techniques, described herein, may be configured as a convenient, easy-to-use, resource for a user to assess various aspects of his or her health and wellness. In some examples, it may be configured as a station or kiosk located in a public, retail, or corporate setting, and the types of assessments, recommendations, advertisements and incentives provided by the system (e.g., the station or kiosk) may be customized according to its location. In other examples, the system may be configured to receive orders and payments for services or products (e.g., using a card reader, as described herein). In some examples, the system may be configured to upload information to track customer usage, demographics, screening results, or other information. Such information also may be tracked using an associated website, web application, mobile application, or the like, wherein additional recommendations (e.g., of services, products, etc.) and incentives (e.g., coupons, advertisements, etc.) may be provided. In other examples, even maintenance information may be tracked to ensure operability and to indicate when repair may be needed. In still other examples, the system may be configured to communicate with other parties that may be located in off-site location, such as the office of a doctor, corporate medical officer, or medical/insurance company.

In some examples, the various computer systems may be configured for use with one or more applications, some of which may be written (i.e., developed) using programming and formatting languages, protocols, and specifications that are native to such systems. The computer systems may be implemented as any type of computing device, hardware, software, firmware, circuitry, or a combination thereof for purposes of providing computational and processing capabilities for the techniques described herein. Any of sub-systems shown and others not shown, may be provided by third parties or organically developed for native implementation (i.e., developed to work with a virtual currency platform).

In some examples, some, none, or all of the logic for the above-described techniques may be implemented as a computer program or application or as a plug-in, module, or sub-component of another application. The described techniques may be varied and are not limited to the examples or descriptions provided. In some examples, applications may be developed for download to mobile communications and computing devices (e.g., laptops, mobile computers, tablet computers, smart phones, etc.), being made available for download by the user either directly from the device or through a website. In other examples, applications may provide health and lifestyle-related information. In some examples, docks or cradles may be implemented to allow users to connect their mobile devices to a Wellness Center to enable the transfer of information associated with a self-screening health assessment.

In still other examples, a user may be motivated to respond in a certain manner or take a desired action by utilizing gaming or gamification techniques. Such techniques may include, but not limited to, tracking and rewarding personal achievements (e.g., a user collecting something, achieving a goal, or completing a task associated with a self-screening health assessment). In other examples, a user interface may be configured to present information associated with a self-screening health assessment providing categories for classifying different users. For example, a user may be a member of a group that shares a common trait, characteristic, or goal. Using group motivation techniques, a given user may be able to influence other user's actions. In other examples, contextual communications may be utilized, wherein a signal or a message (e.g., a progress bar, congratulatory message, etc.) encouraging user behavior appears at a particular time in, or part of, a wellness assessment session.

Although the foregoing examples have been described in some detail for purposes of clarity of understanding, the invention is not limited to the details provided. There are many alternative ways of implementing the invention. The disclosed examples are illustrative and not restrictive.

What is claimed:

1. A device, comprising:
a plurality of monitors configured to display one or more graphics associated with screening vision acuity;
a plurality of mirrors configured to reflect at least one of the one or more graphics along an optical path, the plurality of mirrors enclosed in a chamber, the chamber being moveable:
a view portal opening through which the at least one of the one or more graphics is visible;
a display configured to provide a user interface associated with a wellness assessment;
a touchscreen configured to transmit a user selection to a computer; and
a housing coupled to at least one of the plurality of monitors, the chamber and the touchscreen.

2. The device of claim 1, wherein the plurality of monitors comprises a monitor configured to display a graphic associated with screening distance vision and another monitor configured to display another graphic associated with screening near and central vision acuity.

3. The device of claim 2, wherein the monitor is coupled to the chamber.

4. The device of claim 2, wherein the another graphic comprises an Amsler grid.

5. The device of claim 1, further comprising a lift configured to move the chamber.

6. The device of claim 5, further comprising a brake coupled to the lift.

7. The device of claim 1, wherein the chamber is coupled to the housing using rails along which the chamber is moveable.

8. The device of claim 1, wherein the touchscreen comprises an infrared touchscreen.

9. The device of claim 1, further comprising a sensor configured to sense a distance between a target and the sensor.

10. The device of claim 9, wherein the sensor further is configured to sense the top of a user's head.

11. The device of claim 1, further comprising a microphone configured to receive audio input associated with the wellness assessment.

12. The device of claim 1, further comprising a speaker configured to provide audio output associated with the wellness assessment.

13. The device of claim 1, wherein the computer is network enabled.

14. The device of claim 1, further comprising a card reader configured to obtain identification information from a card.

15. A method, comprising:
positioning a user interface at an eye level, the user interface being displayed on a monitor coupled to a lift;
requesting input associated with a wellness assessment using the user interface;
performing the wellness assessment;
providing a result of the wellness assessment using a display; and
providing a recommendation associated with the result on the display.

16. The method of claim 15, further comprising determining the eye level prior to position the user interface.

17. The method of claim 16, wherein the determining the eye level comprises receiving a sensory input designating the eye level using a touchscreen.

18. The method of claim 16, wherein the determining the eye level comprises receiving a sensory input designating the eye level using a sensor configured to sense a distance between a target and the sensor.

19. The method of claim 15, wherein the wellness assessment comprises a diabetes risk assessment.

20. The method of claim 15, wherein the wellness assessment comprises a vision acuity assessment.

21. The method of claim 20, wherein the performing the wellness assessment comprises displaying a graphic at the eye level for performing the vision acuity assessment.

22. The method of claim 15, wherein the providing the result comprises providing a graphical representation of the result on the display.

23. The method of claim 15, further comprising sending an electronic mail message associated with the result.

24. The method of claim 23, wherein the electronic mail message further is associated with the recommendation.

25. The method of claim 15, wherein the recommendation includes a service provider that provides a service associated with the wellness assessment.

26. The method of claim 15, wherein the recommendation includes a product promotion associated with the wellness assessment.

27. The method of claim 15, further comprising displaying an advertisement associated with the wellness assessment.

28. A computer readable medium including instructions for performing a method, the method comprising:
requesting information associated with a wellness assessment using a user interface;
performing the wellness assessment;
providing a result of the wellness assessment using a display; and
providing a recommendation associated with the result on the display.
